(19) United States
(12) Patent Application Publication
(10) Pub. No.: US 2025/0279185 A1
(43) Pub. Date: Sep. 4, 2025

(54) SYSTEM AND METHODS FOR INTEGRATION OF NETWORK COMPONENTS COMPLYING WITH HL7

(71) Applicant: Aidoc Medical Ltd, Tel Aviv (IL)

(72) Inventors: Ariel PERSIKO, Lod (IL); Michael BRAGINSKY, Rishon LeZion (IL)

(73) Assignee: Aidoc Medical Ltd, Tel Aviv (IL)

(21) Appl. No.: 18/594,140

(22) Filed: Mar. 4, 2024

(51) Int. Cl. G16H 30/20; G16H 10/60

(52) U.S. Cl. CPC G16H 30/20; G06F 16/254; G06F 16/258; G16H 10/60

(57) ABSTRACT

There is provided a method of supporting automatic integration of an interface of a medical component within a medical network, comprising: monitoring a message(s) within the medical network complying with a standard for communication of health related data according to a first set of definitions of segments and sub-segments of the messages, feeding a sub-segment(s) of a segment of the message(s) into a classifier(s) for obtaining a classification category for each of the sub-segments, wherein the classification category is according to a second set of definition of sub-segments of messages destined for the interface of the medical component being integrated within the medical network, generating a mapping dataset according to the classification category obtained for each of the sub-segments, for mapping between the first set and the second set of definitions, and automatically converting sub-segments of messages sent between the medical network and the interface according to the mapping dataset.

### BACKGROUND

The present invention, in some embodiments thereof, relates to integration of interfaces within a network and, more specifically, but not exclusively, to integration of interfaces within a medical network complying with HL7.

HL7, or Health Level 7, is a set of international standards for the exchange, integration, sharing, and retrieval of electronic health information. It provides a framework for the communication of health-related data between different software applications used by healthcare providers. HL7 standards are widely used in the healthcare industry to facilitate interoperability between various health information systems.

### SUMMARY

According to a first aspect, a computer implemented method of supporting automatic integration of an interface of a medical component within a medical network, comprises: monitoring at least one message within the medical network complying with a standard for communication of health related data according to a first set of definitions of segments and sub-segments of the messages, feeding at least one sub-segment of a segment of the at least one message into at least one classifier, obtaining at least one classification category for each of the at least one sub-segments as an outcome of the at least one classifier, wherein the at least one classification category is according to a second set of definition of sub-segments of messages destined for the interface of the medical component being integrated within the medical network, generating a mapping dataset according to the at least one classification category obtained for each of the at least one sub-segments of the at least one message, for mapping between the first set of definitions and the second set of definitions, and automatically converting sub-segments of messages sent between the medical network and the interface according to the mapping dataset.

According to a second aspect, a system for supporting automatic integration of an interface of a medical component within a medical network, comprises: at least one processor executing a code for: monitoring at least one message within the medical network complying with a standard for communication of health related data according to a first set of definitions of segments and sub-segments of the messages, feeding at least one sub-segment of a segment of the at least one message into at least one classifier, obtaining at least one classification category for each of the at least one sub-segments as an outcome of the at least one classifier, wherein the at least one classification category is according to a second set of definition of sub-segments of messages destined for the interface of the medical component being integrated within the medical network, generating a mapping dataset according to the at least one classification category obtained for each of the at least one sub-segments of the at least one message, for mapping between the first set of definitions and the second set of definitions, and automatically converting sub-segments of messages sent between the medical network and the interface according to the mapping dataset.

According to a third aspect, a non-transitory medium storing program instructions for supporting automatic integration of an interface of a medical component within a medical network, which when executed by at least one processor, cause the at least one processor to: monitor at least one message within the medical network complying with a standard for communication of health related data according to a first set of definitions of segments and sub-segments of the messages, feed at least one sub-segment of a segment of the at least one message into at least one classifier, obtain at least one classification category for each of the at least one sub-segments as an outcome of the at least one classifier, wherein the at least one classification category is according to a second set of definition of sub-segments of messages destined for the interface of the medical component being integrated within the medical network, generate a mapping dataset according to the at least one classification category obtained for each of the at least one sub-segments of the at least one message, for mapping between the first set of definitions and the second set of definitions, and automatically convert sub-segments of messages sent between the medical network and the interface according to the mapping dataset.

In a further implementation form of the first, second, and third aspects, the standard for communication of health related data comprises Health Level 7 (HL7), and the segments and sub-segments are defined by HL7.

In a further implementation form of the first, second, and third aspects, the at least one classifier is trained on a training dataset of a plurality of records, wherein a record includes a sub-segment of a segment of a message according to the first set of definitions and a ground truth label according to the second set of definitions.

In a further implementation form of the first, second, and third aspects, the record further includes an index of the sub-segment indicating sequential position within a plurality of sub-segments of the segment of the message.

In a further implementation form of the first, second, and third aspects, the record further includes at least one additional sub-segment preceding and/or following the sub-segment according to a sequence of sub-segment defined for the segment.

In a further implementation form of the first, second, and third aspects, a plurality of classifiers are trained, each classifier trained on a different training dataset for a different respective segment of a plurality of segments of the message, each training dataset including a plurality of records, wherein a record includes a sub-segment of the respective segment of a message according to the first set of definitions and a ground truth label according to the second set of definitions.

In a further implementation form of the first, second, and third aspects, further comprising: parsing each message into a plurality of segments, for each respective segment of the plurality of segments of the message: identifying a type of a plurality of types for the respective segment, selecting a classifier of a plurality of classifiers according to the type of the respective segment, wherein each classifier is trained for generating the at least one classification category in response to an input of at least one sub-segment of the segment satisfying a specific type of the plurality of types, wherein a plurality of classifiers are trained for the plurality of segment types, wherein the respective sub-segment of the segment is fed into the selected classifier.

In a further implementation form of the first, second, and third aspects, feeding comprises feeding a combination of a sub-segment of a segment of the message and an index of the sub-segment indicating sequential position within a plurality of sub-segments of the segment of the message.

In a further implementation form of the first, second, and third aspects, feeding comprises feeding a combination of a sub-segment of the message and at least one additional sub-segment preceding and/or following the sub-segment according to a sequence of sub-segments of a segment of the message.

In a further implementation form of the first, second, and third aspects, the outcome of the at least one classifier fed a sub-segment of the first set of definitions comprises a plurality of probabilities for a plurality of sub-segments based on the second set of definitions, wherein the at least one classification category for the sub-segment fed into the at least one classifier is computed by applying a process to the plurality of probabilities of the plurality of sub-segments, wherein the process is fed a matrix of the plurality of probabilities and outputs a unique classification category for all non-zero sub-segments.

In a further implementation form of the first, second, and third aspects, the process selects a definition from the second set having a highest probability, wherein the at least one classification category comprises the selected definition.

In a further implementation form of the first, second, and third aspects, the process selects a definition from the second set having a highest probability when a difference between the highest probability and second highest probability is greater than a threshold.

In a further implementation form of the first, second, and third aspects, further comprising: in response to an indicating that no valid classification of the sub-segment is determined, generating a presentation on a display for manual classification of the sub-segment by a user.

In a further implementation form of the first, second, and third aspects, the monitoring, the feeding, the obtaining, and the generating are performed during a set-up phase where the monitoring is performed on messages sent between existing network connected components over the medical network excluding the interface, and the automatic conversion is performed dynamically in real-time for messages exchanged between the medical network and the interface.

In a further implementation form of the first, second, and third aspects, further comprising iterating the set-up phase periodically at a plurality of time intervals and/or in response to an event, for regenerating the mapping dataset and/or detecting changes in the mapping dataset.

In a further implementation form of the first, second, and third aspects, the monitoring, the feeding, the obtaining, and the generating are performed in real-time for each message exchanged between the medical network and the interface for dynamic computation of a mapping dataset for each message, wherein each message is dynamically converted in real-time using the mapping dataset computed for each respective message.

In a further implementation form of the first, second, and third aspects, the interface comprises an input into a machine learning model.

In a further implementation form of the first, second, and third aspects, further comprising, for each message: dividing each segment of a plurality of segments of the message into a plurality of sub-segments, converting each sub-segment into a string, wherein feeding comprises feeding the string.

In a further implementation form of the first, second, and third aspects, components connected to the medical network are selected from: electronic medical record (EMR) dataset, imaging device, and AI application.

In a further implementation form of the first, second, and third aspects, the medical network comprises an integration target and the medical component comprises an integration source.

In a further implementation form of the first, second, and third aspects, the second set of definitions are of a translator process, and the interface of the medical component complies with a third set of definitions, wherein the mapping dataset comprises a first mapping dataset for mapping between the first set of definitions of the medical network and the second set of definitions of the translator, and further comprising performing the monitoring for at least one message according to the third set of definitions, the feeding into at least one second classifier, the obtaining from the at least one second classifier, and the generating a second mapping dataset for mapping between the second set of definitions of the translator process and the third set of definitions of the interface, wherein automatically converting comprises automatically converting messages sent between the medical network and the interface according to the first mapping dataset and the second mapping dataset, by converting from the first definition to the second definition, and from the second definition to the third definition, and/or from the third definition to the second definition, and from the second definition to the first definition.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of components of a system for supporting automatic integration of an interface of a medical component within a medical network, in accordance with some embodiments of the present invention;

FIG. 2 is a flowchart of a method of supporting automatic integration of an interface of a medical component within a medical network, in accordance with some embodiments of the present invention;

FIG. 3 is a flowchart of a method of training one or more classifiers that determine a definition for a sub-segment of a message according to a standard for communication of health related data, in accordance with some embodiments of the present invention;

FIG. 4 is an example of a segment of a message complying with HL7, and parsed sub-segments 404, in accordance with some embodiments of the present invention;

FIG. 5 is an example of a matrix of probabilities for generating a mapping dataset for mapping between sub-segments of the first and second set of definitions, in accordance with some embodiments of the present invention;

FIG. 6 is another example of a matrix of probabilities for generating a mapping dataset for mapping between sub-segments of the first and second set of definitions, in accordance with some embodiments of the present invention;

FIG. 7 is an example of a mapping dataset, in accordance with some embodiments of the present invention;

FIG. 8 is another example of a matrix of probabilities for generating a mapping dataset for mapping between sub-segments of the first and second set of definitions, in accordance with some embodiments of the present invention; and

FIG. 9 is an example of another mapping dataset, in accordance with some embodiments of the present invention.

### DETAILED DESCRIPTION

The present invention, in some embodiments thereof, relates to integration of interfaces within a network and, more specifically, but not exclusively, to integration of interfaces within a medical network complying with HL7.

As used herein the term “standard” is short for standard for communication of health related data. The standard may refer to HL7, or other suitable standards and/or protocols. The term standard may refer to a protocol.

As used herein, the medical component having an interface, which is being automatically integrated, may be referred to as an integration source. The medical network, which includes other network connected components, into which the medical component is being integrated, may be referred to herein as an integration target.

As used herein, the term medical network is meant as a not necessarily limiting example. A device (e.g. imaging equipment, medical diagnostic) and/or process (e.g., ML model, AI application) and/or dataset (e.g., electronic health record database) may be substituted for the medical network, for example, an electronic database for storing images may be integrated with an imaging device that generates the images.

As used herein, the term automatic integration may refer to a semi-automatic integration process, in which some features are automatically performed and some features may be manually performed. For example, the mapping dataset may be automatically generated as described herein, however, some manual user intervention may be required, for example, in certain cases that cannot be accurately resolved automatically.

An aspect of some embodiments of the present invention relates to systems, methods, devices, and/or code instruction (stored on a data storage device and executable by one or more processors) for supporting automatic integration of an interface of a medical component within a medical network. The medical component may be, for example, a machine learning (ML) model and/or artificial intelligence (AI) application, an imaging device, a diagnostic device, a database (e.g., electronic health records), and the like. The medical network complies with a standard for communication of health related data, for example, health level 7 (HL7). The standard is implemented according to a first set of definitions of segments and sub-segments for messages sent over the medical network. Messages within the medical network are monitored. At least one sub-segment of a segment of the message(s) are fed into a classifier. A classification category is obtained for each of the sub-segments as an outcome of the classifier. The classification category is according to a second set of definition of sub-segments of messages destined for the interface of the medical component being integrated within the medical network. The second set of definitions may be different than the first set of definitions. A mapping dataset that maps between the first set of definitions and the second set of definitions is automatically generated according to the classification category obtained for each of the sub-segments of each segment of the message(s). Sub-segments of messages sent between the medical network and the interface may be automatically converted according to the mapping dataset. The mapping dataset may enable automatic integration of the medical component within the medical network.

The second set of definitions may be according to the standard, optionally the same standard that defines the first set of definitions used by the existing components of the medical network, optionally HL7. The problem is that since there is some ambiguity in interpretation of the standard, such as different healthcare networks interpreting the same standard (e.g., HL7) in different ways, the sub-segments of the existing components of the medical network may not directly map to what the interface of the medical component being integrated into the medical network expects. The interface of the medical component expects the sub-segments of the message to comply with the second set of definitions, whereas the sub-segments of the message comply with the first set of definitions.

At least some embodiments described herein address the technical problem of integration of interfaces within a network, for example, of an application being added to a network for integration with other application and/or devices within the network. In particular, the technical problem relates to integration of medical interfaces within a medical network, for example, between medical databases, medical images, and medical devices generating medical data. The medical interfaces comply with a standard for communication of health data, optionally HL7. At least some embodiments described herein improve the technology of tools for improving integration of interfaces. In particular, within a medical network complying with HL7. At least some embodiments described herein improve upon existing approaches for integration of interfaces. In particular, within a medical network complying with HL7.

With the massive growth in the number of healthcare applications there is a growing demand for interoperability between different applications. In particular, it is essential to ensure that different applications are connected to the EMR (Electronic Medical Records) systems where patient and clinical data is stored. Typically, this is done via the standard HL7 protocol that defines what information is stored in every segment/sub-segment of the device input and output. However, HL7 is a meta-standard rather than rigid standard. In other words, each user/hospital adopts different sub-segment definitions. HL7 segments tend to be well defined, such that all the users and/or network connected components use the same convention. There is generally no confusion in this domain. The technical problem is how to parse a given segment into the consistent sub-segments. Accordingly, every time a new healthcare application is to be integrated, the integration team needs to define a translation table between the convention adopted by the given application and that used by the hospital.

Moreover, HL7 sub-segment definitions may change over time. This will cause misalignment. As a result, recalibration of the translation tables would be required. Presently, detection and correction of misalignments is done manually. It is a tedious and costly process.

This problem is further exacerbated in automatic applications (such as AI) where errors in field/sub-field definitions may be difficult to detect. Users may notice system efficacy deterioration without knowing the reason.

Typically, configuration of the HL7 sub-segments is done manually. The parties that configure the system exchange specs and accordingly the systems are configured.

For example, the HL7 standard calls for a patient ID number to appear at the second sub-segment of the PID segment of the HL7 message. However, some hospitals use this sub-segment for the patient's name, while patient ID number is deferred to the sub-segment number six.

IT experts performing integration will manually inspect subfield definitions and determine translation tables.

Sometimes, specs are ambiguous. IT experts may resort to the examination of the actual content (in our example, looking at the content of the subfield six, the expert will understand that, at the given site, subfield six contains patient ID number, and associate it with the subfield two of the standard).

The process is laborious and error prone. Indeed, HL7 standard describes 130 segments having tens of sub-segments each. Hence, thousands of definitions must be reviewed and adjusted. As a result, integration of new systems becomes a lengthy and costly process. In some cases, integration costs may exceed application costs.

At least some embodiments described herein relate to approaches for using AI (Artificial Intelligence) for automatic identification of the HL7 sub-segments. These approaches are designed to solve the aforementioned bottleneck enabling faster and cheaper healthcare systems integration.

Moreover, the same approach can be used for monitoring system operation to detect and correct changes as they occur during normal operation.

The classifier described herein may be trained on a large number of manually classified HL7 messages collected from a wide variety of users. After the training, for each HL7 segment, the classifier may provide correct classification of all the sub-segments.

Using embodiments described herein, classification of the HL7 is automated, fully or mostly. There is not necessarily a need for manual sub-segment identification, apart for example for a few cases where classification is inaccurate.

Embodiments described herein may provide one or more of the following potential advantages:
Integration costs are reduced significantly.
Integration will be done much, much faster.
The number of erroneous identifications may be reduced.
Definition changes can be identified and corrected on-line.

At least some embodiments described herein improve the aforementioned technical problem, and/or improve the aforementioned technical field and/or improve upon the aforementioned prior approaches, by generating a mapping dataset for mapping between a first set of definitions used by an integration target (e.g., medical network) and second set of definitions used by an integration source (e.g., medical component). The first and second set of definitions are for defining sub-segments of segments of messages that comply with a standard for communication of health related data, optionally HL7. The mapping dataset is generated according to classification categories obtained for sub-segments of messages, for example, being monitored on the medical network. The classification categories are obtained as an outcome of a classifier fed sub-segments extracted from each segment of the messages(s).

Embodiments described herein may be used at the beginning of integration. The site into which the medical component is being integrated may provide a few days of data. The classifier is used to build the mapping between the site data and the correct category of each data position used by the new application to be integrated, as described herein. Once such mapping is established, the integration process may process. Some embodiments may be for use in an online mode, i.e., when a new message is obtained, the classifier may be used to define the category of each sub-segment “on the flight”. A potential advantage of the online approach is that, if there is a change in the order of sub-segments, the change is identified in-time, which may prevent or reduce performance degradation. The on-line mode of operation may relate to creating a Universal HL7 Translator (UHT). The devices of the medical network (e.g., EMR, different imaging devices, and different AI applications) may communicate with each other through this UHT system that translates the messages to the “language” of the given user. In some embodiments the two options may be combined by defining the mapping as in the first option but, in the production, the classifier may be run from time to time. A search for a contradiction between the fixed mapping and the classifier output may be performed to determine whether the mapping is to be updated.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

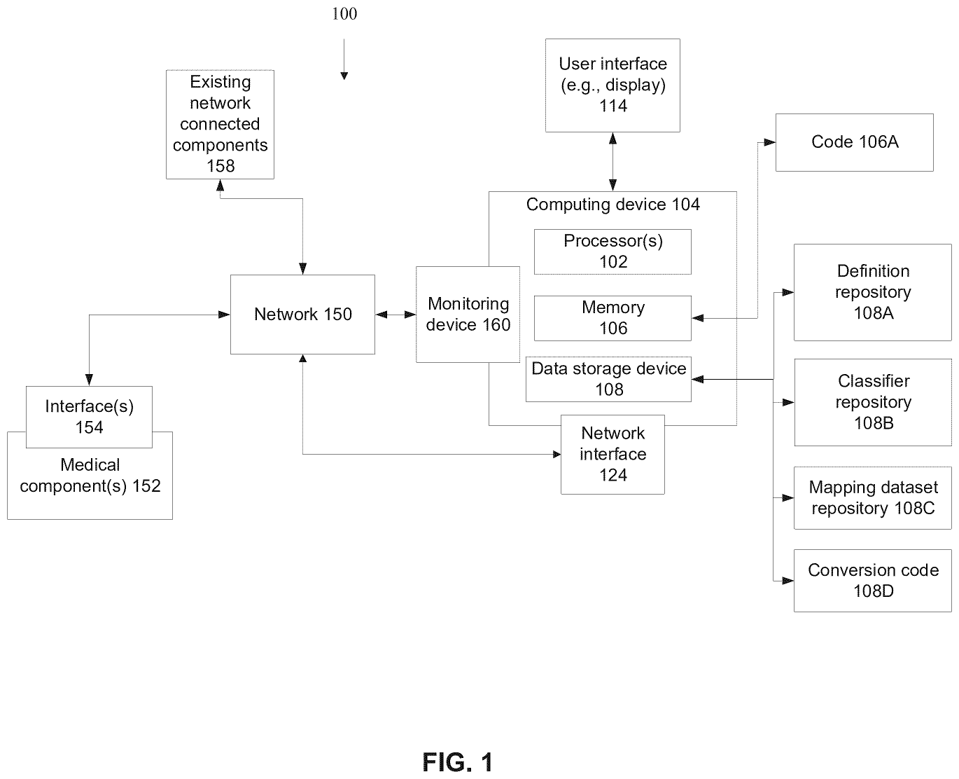

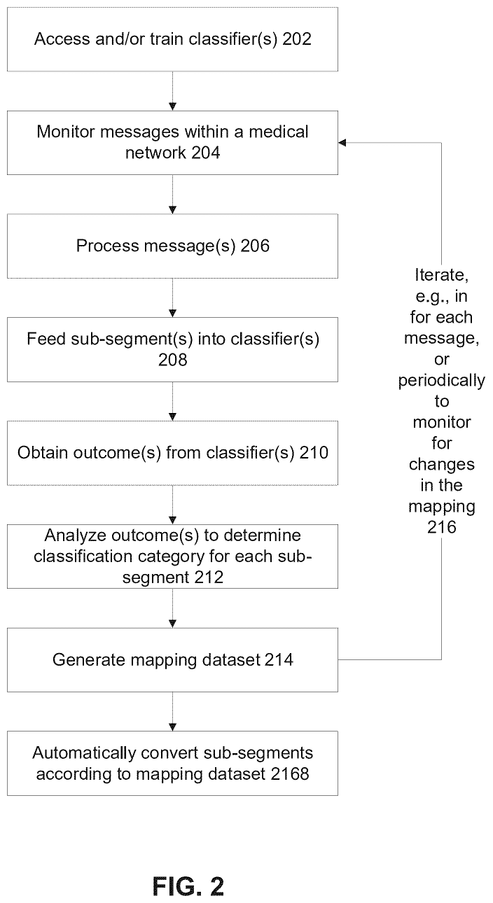

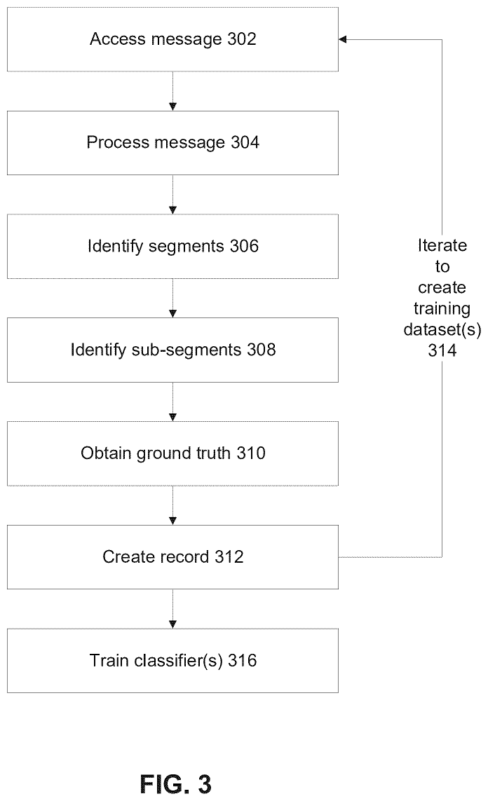

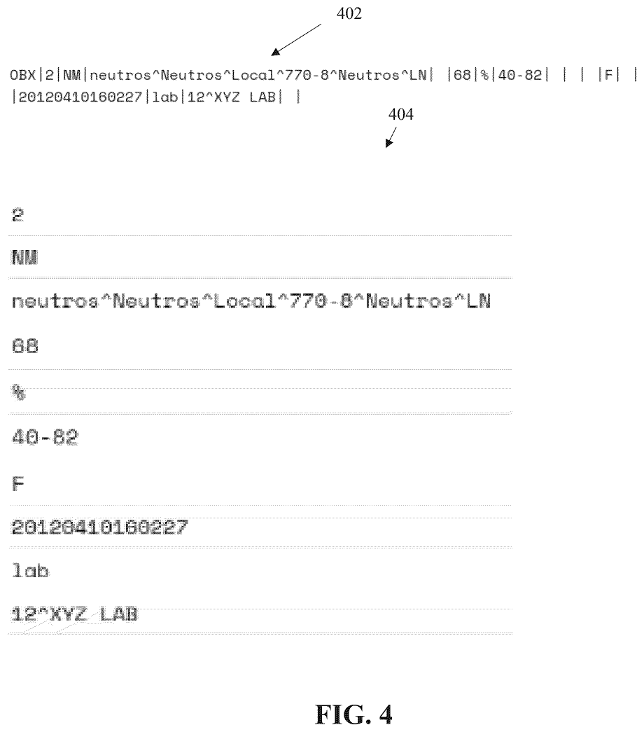

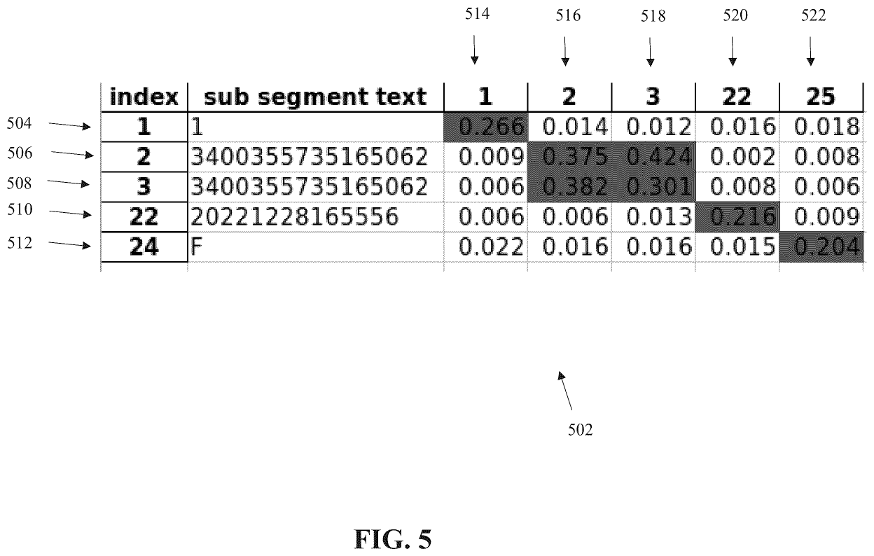

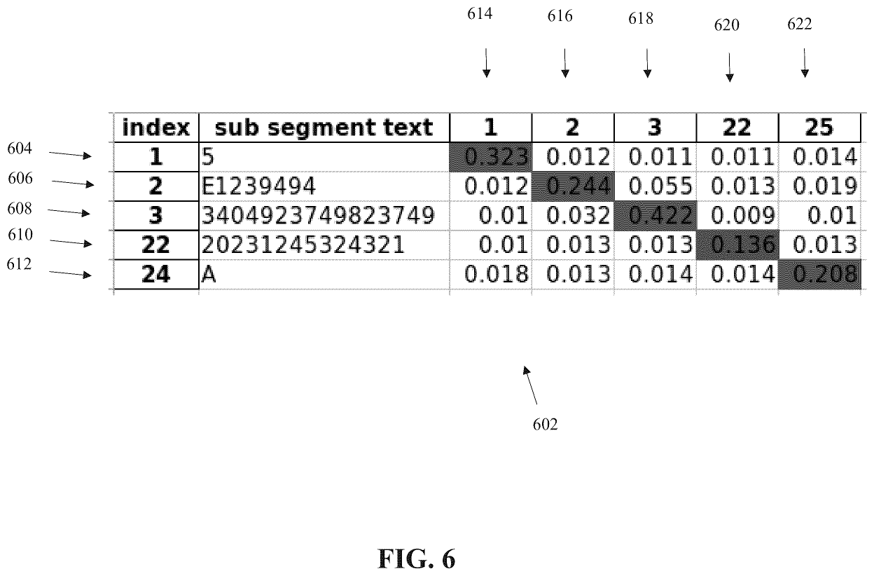

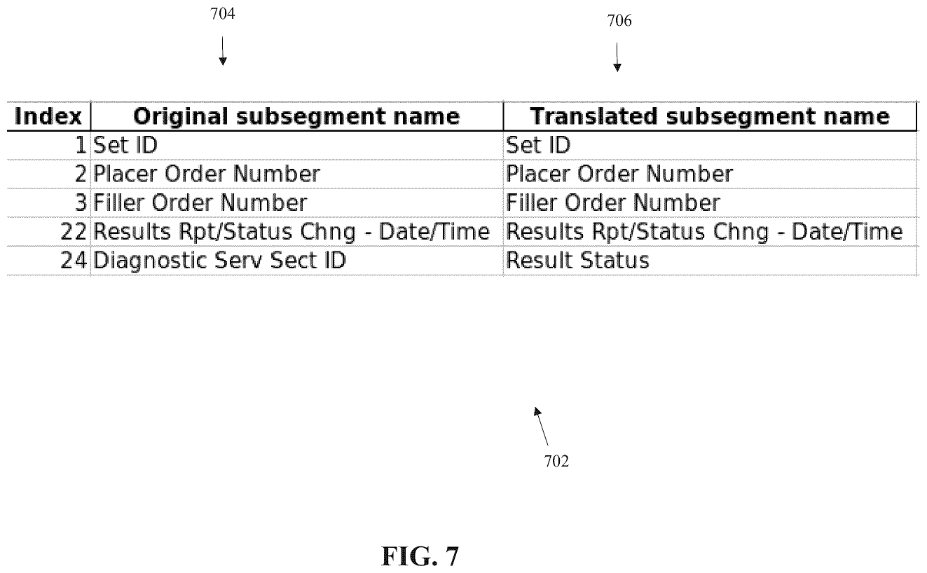

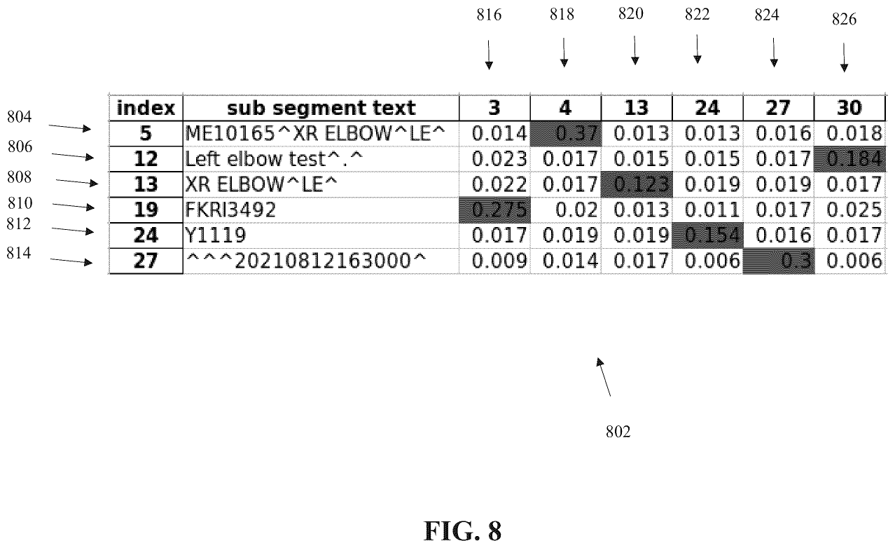

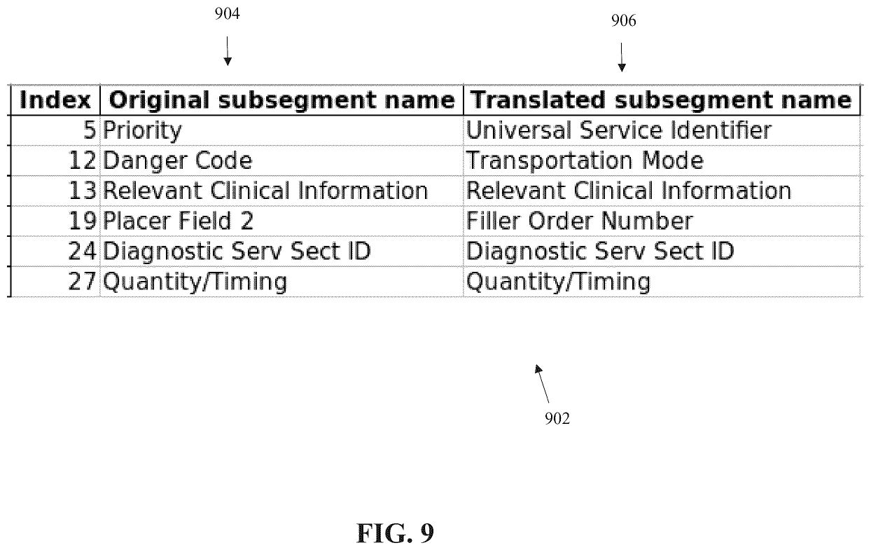

Reference is made to FIG. 1, which is a block diagram of components 100 of a system for supporting automatic integration of an interface of a medical component within a medical network, in accordance with some embodiments of the present invention. Reference is also made to FIG. 2, which is a flowchart of a method of supporting automatic integration of an interface of a medical component within a medical network, in accordance with some embodiments of the present invention. Reference is also made to FIG. 3, which is a flowchart of a method of training one or more classifiers that determine a definition for a sub-segment of a message according to a standard for communication of health related data, in accordance with some embodiments of the present invention. Reference is also made to FIG. 4, which is an example of a segment 402 of a message complying with HL7, and parsed sub-segments 404, in accordance with some embodiments of the present invention. Reference is also made to FIG. 5, which is an example of a matrix of probabilities 502 for generating a mapping dataset for mapping between sub-segments of the first and second set of definitions, in accordance with some embodiments of the present invention. Reference is also made to FIG. 6, which is another example of a matrix of probabilities 602 for generating a mapping dataset for mapping between sub-segments of the first and second set of definitions, in accordance with some embodiments of the present invention. Reference is also made to FIG. 7, which is an example of a mapping dataset 702, in accordance with some embodiments of the present invention. Reference is also made to FIG. 8, which is another example of a matrix of probabilities 802 for generating a mapping dataset for mapping between sub-segments of the first and second set of definitions, in accordance with some embodiments of the present invention. Reference is also made to FIG. 9, which is an example of another mapping dataset 902, in accordance with some embodiments of the present invention.

Referring now back to FIG. 1, system 100 may implement the acts of the method described with reference to FIGS. 2-9, by processor(s) 102 of a computing device 104 executing code instructions (e.g., code 106A) stored in a memory (also referred to as a program store).

Computing device 104 may monitor packets (and/or other formats of messages) transmitted over a network 150 (e.g. medical network) between existing network connected components 158, and/or between an interface(s) 154 of a medical component 152 and one or more of existing network connected components 158.

Medical component 152 may be in the process of being integrated into network 150, and/or may be previously integrated. Interface(s) 154 of medical component 152 may be, for example, a virtual interface, an application programming interface (API), an input into a machine learning model, an input into a database such as an electronic medical record (EMR) database, and an input into a picture archiving and communication system (PACS) server, and the like.

Examples of existing network connected components 158 and/or medical component 152 include: machine learning (ML) models (also referred to herein as artificial intelligence (AI) application), EMR databases, and medical devices such as medical imaging devices such as devices that support a patient (e.g., ventilator, heart-lung machine), devices that monitor the patient (e.g., ECG, blood pressure), devices that analyze tissue of the patient (e.g., blood gas analyzer), and the like.

Computing device 104 is programmed and/or positioned within and/or in communication with network 150 to monitor and/or intercept packets (and/or other formats) of messages, optionally compliant with HL7, transmitted over network 150 between existing network connected components 158 and/or between existing network connected components 158 and interface(s) 154 of medical component(s) 152. Computing device 104 may be located, for example, in a server farm in communication with network 150, within a central switch of network 150 through which all packets flow, within interface 154, within a computing cloud, and/or other locations.

Computing device 104 may include and/or be in communication with a network monitoring device 160 for monitoring packet traffic (and/or other formats) within network 150 compliant with the HL7 standard (or another standard), for example, a packet sniffer, a packet analyzer, a network sensor, and/or network gateway.

Optionally, computing device 104 monitors and/or intercepts all packets transmitted over network 150 compliant with HL7 (or another standard). It is noted that computing device 104 may be distributed among multiple devices at different locations in network 150 to monitor and/or intercept all the packets. Alternatively, computing device 104 is installed in a single location, where traffic exiting and/or entering medical device(s) 152 is accessible from the single location.

Computing device 104 may be implemented as, for example one or more and/or combination of: a router, a switch, a network administration server, a group of connected devices, a client terminal, a server, a virtual server, a computing cloud, a virtual machine, a desktop computer, a thin client, a network node, a network server, and/or a mobile device.

Hardware processor(s) 102 of computing device 104 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include a single processor, or multiple processors (homogeneous or heterogeneous) arranged for parallel processing, as clusters and/or as one or more multi core processing devices.

Memory 106 stores code instructions executable by hardware processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Memory 106 stores code 106A that implements one or more features and/or acts of the method described with reference to FIGS. 2-9 when executed by hardware processor(s) 102.

Computing device 104 may include data storage device(s) 108 for storing data, for example, one or more of: a definition repository 108A set for storing definitions for segments and/or sub-segments of HL7 of the existing network connected components 158 and/or of interface(s) 154, a classifier repository 108B set for storing one or more classifiers that classify sub-segments, a mapping dataset repository 108C set for storing one or more computed mapping datasets that map between the first definition used by the existing network connected components 158 and the second definition used by the interface(s) 154 of medical component(s) 152, and/or conversion code 108D for converting between messages of the first definition used by the existing network connected components 158 and the second definition used by the interface(s) 154 of medical component(s) 152, as described herein. Data storage device(s) 108 may be implemented as, for example, a memory, a local hard-drive, virtual storage, a removable storage unit, an optical disk, a storage device, and/or as a remote server and/or computing cloud (e.g., accessed using a network connection).

Network 150 may be implemented as, for example, a private network, a local area network, a wireless network, a wired network, the internet, a virtual network, a virtual private network, a cellular network, and/or combinations of the aforementioned. For example, network 150 is a medical network installed for communicating with a healthcare organization and/or between healthcare organizations, such as in a hospital, ICU, ward, radiology clinic, and/or other healthcare setting, for connecting devices of the healthcare provider.

Computing device 104 includes and/or is in communication with one or more physical user interfaces 114 that include a mechanism for user interaction, for example, to enter data such as manual classification of sub-segments when automated classification is invalid and/or for viewing data such as the automatically computed mapping dataset.

Exemplary physical user interfaces 114 include, for example, one or more of, a touchscreen, a display, gesture activation devices, a keyboard, a mouse, and voice activated software using speakers and microphone.

Computing device 104 may include a network interface 124 for connecting to network 150, for example, one or more of, a network interface card, a wireless interface to connect to a wireless network, a physical interface for connecting to a cable for network connectivity, a virtual interface implemented in software, network communication software providing higher layers of network connectivity, and/or other implementations. It is noted that the network interface 124 of computing device 104 may be integrated with network monitoring device 160.

Referring now back to FIG. 2, at 202, one or more classifiers may be accessed and/or trained. An exemplary approach for training the classifier(s) is described with reference to FIG. 3.

At 204, one or more messages within a medical network are monitored.

Messages and/or devices and/or components (e.g., software running on a computer) connected to the medical network comply with a standard for communication of health related data, for example, Health Level 7 (HL7).

An interface of a medical component is being integrated within the medical network, and/or the interface has been integrated within the medical network. Examples of medical components being integrated include a machine learning model, an artificial intelligence (AI) application, an imaging device, a diagnostic device, a database (e.g., EMR), and the like. Examples of existing components and/or devices that are connected to the medical network include a machine learning model, an artificial intelligence (AI) application, an imaging device, a diagnostic device, a database (e.g., EMR), and the like.

The monitoring may be performed on messages between existing devices connected to the medical network (excluding the interface) and/or on messages sent between existing devices connected to the medical network and the interface.

The monitoring may be performed, for example, by a packet sniffer that sniffs packets traversing the network, a packet extractor that extracts packets from the network, by a gateway through with all packets or relevant packets (e.g., travelling to and/or from the interface) pass, and the like.

At 206, the monitored packets may be processed.

The processing of the monitored packets may include parsing each message into multiple segments, for example, a segment per line, and/or divided by delimiters. The segments may be defined according to the standard, optionally HL7.

A type of each segment may be identified. The type may be according to the standard, optionally HL7. For example, the first 3 characters (e.g., string) of each line representing each segment indicate the type according to HL7. Examples of types of segments include: DG1 denoting diagnosis, EVN denoting event type, MSH denoting message header, OBR denoting observation request, OBX denoting observation result, PID denoting patient identification, and the like.

The processing may further included dividing (e.g., parsing) each segment into multiple sub-segments, where the actual information may reside. For example, for the OBX segment, the following sub-segments may be defined: value type, observation identifier, units, abnormal flags, etc.

It is noted that in standards that define a hierarchy, levels lower than sub-segments may be flattened and treated as sub-segments.

Each sub-segment may be identified by being separated by a delimiter.

Delimiters indicating segments and/or sub-segments may be defined by the standard, optionally HL7, for example, vertical bar (|), caret (\hat{ }), ampersand (&), number sign (#), tilde (˜), and the like.

Each segment and/or sub-segment may be converted into a string.

Each sub-segment may be assigned an index indicating its location in a sequential ordering of sub-segments within a respective segment. Different types of segments may be defined to include different sub-segments and/or different orders of sub-segments.

Each segment and/or sub-segment conforms to a first set of definitions defined by the standard, optionally HL7. The first set of definitions may define what data is to be found in what sub-segment of each type of segment, and/or the expected order of the sub-segments.

At 208, for each segment of the message, one or more sub-segment of a respective segment are fed into a classifier.

Optionally, one or more sub-segments of each respective segment are fed into a respective classifier. The respective classifier may be selected and/or accessed for each segment of the message. For example, the HL7 protocol defines about 130 types of segments. Multiple classifiers corresponding to the multiple types of segments may be accessed and/or trained. A respective classifier has been trained for each type of segment. The respective classifier corresponding to each type of segment may be selected and/or accessed. Each classifier is trained for generating an outcome (e.g., classification category, or probability for each classification category) in response to an input of one or more sub-segments of the respective.

Alternatively, each respective segment is fed as a whole into the corresponding classifier. The segment may include all of the sub-segments therein. Each classifier may be trained for analyzing a certain segment that includes all sub-segments of the segment.

Alternatively, multiple segments, optionally the entire message, may be fed into a main classifier. The classifier may be fed multiple sub-segments from multiple segments of the message. Such classifier may be trained for analyzing a message as a whole, or for analyzing multiple segments of the message.

The string representation of each segment and/or each sub-segment may be fed into the classifier.

Optionally, individual sub-segments of each segment are fed into the classifier (e.g., the selected classifier corresponding to each respective type of segment). For example, each individual sub-segment is sequentially fed into the classifier. Alternatively, a sequence of multiple sub-segments is fed into the classifier, for example, the sequence of all sub-segments of the segment.

In another example, for a current sub-segment being evaluated, one or more sub-segments preceding the current sub-segment and/or one or more sub-segments following the current sub-segment, may be included. For example, a sliding window of size two or more sub-segments may be moved along the sub-segments, where the contents of the sliding window are sequentially fed into the classifier.

Optionally, the sub-segment (optionally each sub-segment in the case of multiple sub-segments) is fed into the classifier in combination with an index indicating sequential position of the sub-segment within the sequence of sub-segments of the segment of the message. The index may be, for example, a number, starting from 1, sequentially allocated to each sub-segment of the segment.

At 210, one or more outcomes are obtained from the classifier for each sub-segment of each segment of the message.

Optionally, the outcome(s) includes a classification category assigned to each sub-segment fed into the classifier. The classification category may be defined according to the second set of definition for sub-segments of messages destined for the interface of the medical component being integrated within the medical network. In such a case, no further analysis may be required.

The second set of definitions may be according to the standard, optionally the same standard that defines the first set of definitions used by the existing components of the medical network, optionally HL7. The problem is that since there is some ambiguity in interpretation of the standard, such as different healthcare networks interpreting the same standard (e.g., HL7) in different ways, the sub-segments of the existing components of the medical network may not directly map to what the interface of the medical component being integrated into the medical network expects. The interface of the medical component expects the sub-segments of the message to comply with the second set of definitions, whereas the sub-segments of the message comply with the first set of definitions.

Alternatively or additionally, the outcomes of the classifier(s) for each inputted sub-segment include a respective probability for each of multiple categories, optionally for all available categories. For example, for a segment in which there are 50 defined sub-segments, for an input of a certain sub-segment (e.g., sub-segment having index number 30/50), probability of sub-segment #30 (according to the first definition) being any one of sub-segments 1-50 (according to the second definition) is computed. 50 probabilities are computed for the input sub-segment, i.e., a respective probability for each possible sub-segment according to the second definition.

For example, in response to an input of sub-segments of a certain type of HL7 segment, a matrix of probabilities is generated based on the outcome of the classifier. The number of rows in the matrix may equal the number of sub-segments in the input segment. The number of columns in the matrix may be equal to the number of possible sub-segments (as defined in the HL7 standard). Entry ij of the matrix may be equal to the probability that input sub-segment i belongs to the classification category j.

Optionally, when the probability matrix (or other representation) is computed, the probability matrix may be transformed into a decision vector providing unique classification for each input sub-segment. Alternatively or additionally, a composite decision (CD) vector may be computed. The CD vector may provide a decision vector for a stream of possible segment inputs, where the assumption is that all the sub-segments arrive in the same order according to the first definition.

At 212, the outcome(s) for each sub-segment may be analyzed.

In the case of the classifier outputting multiple probabilities, the analysis to identify a single category for the sub-segment may include applying a process to the probabilities.

The process may be implemented as, for example, a set of rules, a trained decision making model, a machine learning model, and the like.

The process may include, for example, selecting the highest probability, and setting the category as the sub-segment defined by the second set as the category for the input sub-segment defined by the first set. For example, in the case of the matrix of probabilities described herein, the process assigns j to the row classification if pij is the maximal value in the row i and if pij is larger than a certain predetermined threshold t1.

In another example, the process may include finding two candidate categories for a certain sub-segment, where the two candidate categories correspond to the two highest probabilities. The category with highest probability is assigned to the certain sub-segment when a difference between the highest probability and second highest probability (corresponding to the two candidate categories) is greater than a threshold. For example, finding, in row i, two maximal values pij and pik. Row i is classified as belonging to the category j iff (if and only if) pij−pik>t2, where t2 is a certain predefined positive threshold.

Optionally, the classifier fails to output a valid classification category for the sub-segment, and/or the analysis fails to identify a single classification category. For example, application of the process fail to identify the single classification category.

Optionally, the process may classify two sub-segments into the same category. In these cases, a conflict resolution process may be activated, for example, by following a most likelihood principle.

In response to failure to identify the single classification category, a user interface may be presented on a display of a computer, the user interface being designed to obtain a manual classification category of the sub-segment by a user. Alternatively, in response to failure to identify the single classification category, the sub-segment is assigned the classification category corresponding to the index of the sub-segment within the segment, i.e., the outcome is the same as the input.

At 214, a mapping dataset is automatically generated using the identified classification categories for each sub-segment.

The mapping dataset may include a mapping for each sub-segment of each segment, for example, a respective mapping dataset is generated for each type of segment, or the mapping dataset is generated for multiple types of segments.

The mapping dataset maps between the first set of definitions (e.g., used by the existing components of the medical network) and the second set of definitions (e.g., used by the interface of the medical component being integrated and/or which has been integrated).

At 216, one or more features described with reference to 204-214 may be iterated.

Optionally, features 204-214 are performed during a set-up phase where the monitoring (e.g., as described with reference to 204 of FIG. 2) is performed on messages sent between existing network connected components over the medical network, optionally excluding the interface. During the set-up phase, the mapping dataset may be generated based on multiple monitored messages, optionally between different existing network connected devices. The set-up phase may generate a more accurate mapping dataset.

The set-up phase may be periodically re-run, for updating the mapping dataset and/or for regenerating the mapping dataset and/or detecting changes in the mapping dataset. For example, over time, the first set of definitions may drift, in which case, the mapping dataset is to be regenerated to reflect the changes. The set-up phase may be re-run, for example, every defined time interval and/or in response to an event associated with changes in the definitions (e.g., change of IT staff, introduction of new components into the medical network, and the like).

Alternatively, features described with reference to 204-214 are performed in real-time for each message exchanged between the medical network and the interface, for dynamic computation of a respective mapping dataset for each message.

At 218, sub-segments of messages sent between the medical network and the interface are automatically converted according to the mapping dataset. The conversion may be done by creating an adapted message by changing the order of the current sub-segments that were defined by the first set of definitions, to comply with the second set of definitions according to the mapping dataset. In another example, the message may not be adapted, but rather, the mapping dataset may be fed into the interface optionally in combination with the message, to enable the interface to correctly extract values from the sub-segments using the mapping dataset.

The conversion may be performed dynamically, in real-time or near real-time, for messages exchanged between the medical network and the interface of the medical component being integrated and/or which has been integrated.

Optionally, features described with reference to FIG. 2 may be adapted for creating a translator process that uses a first mapping dataset for converting between messages (including segments and/or sub-segments) of the medical network complying with the first set of definitions, and a second set of definitions of the translator process. The translator process further uses a second mapping dataset for converting between messages (including segments and/or sub-segments) of the interface of the medical component complying with a third set of definitions, and the second set of definitions. In order to convert a message sent from the medical network to the interface, the first mapping dataset is used to convert the sub-segments of the message from the first set of definitions to the second set of definitions. The second mapping dataset is used to convert the sub-segments of the message from the second set of definitions to the third set of definitions. In order to convert a message sent from the interface to the medical network, the second mapping dataset is used to convert the sub-segments of the message from the third set of definitions to the second set of definitions. The first mapping dataset is used to convert the sub-segments of the message from the second set of definitions to the first set of definitions. Features described with reference to FIG. 2 may be adapted according to the first, second, and third set of definitions, using two classifiers trained using adapted training datasets, for computing the first and second mapping datasets.

Referring now back to FIG. 3, at 302, a message sent between components of a medical network is obtained. The message includes segments and sub-segments defined by the standard, optionally HL7, is obtained.

Additional details of obtaining the message may be described, for example, with reference to 204 of FIG. 2.

At 304, the message is processed.

The message may be parsed into multiple segments. Each segment may be divided into multiple sub-segments.

Each sub-segment may be converted into a string.

Additional examples of processing the messages are described, for example, with reference to 206 of FIG. 2.

At 306, a type may be identified for each segment. The identified types are according to the standard.

Additional examples of processing the segments are described, for example, with reference to 206 of FIG. 2.

At 308, for each segment, respective sub-segments are identified according to the standard. The identification of the sub-segments define the first set of definitions.

Optionally, an index indicating the sequential location of each sub-segment in a sequence of sub-segments of the segment is identified.

Additional examples of processing the sub-segments are described, for example, with reference to 206 of FIG. 2.

At 310, a ground truth label is assigned to each sub-segment according to the second set of definitions. The ground truth label may be created, for example, manually by a user visually inspecting the contents of the sub-segments, such as within a user interface presented on a display of a client terminal. The ground truth may be automatically created, for example, by code designed to analyze the sub-segments.

At 312, a record is created for the sub-segment.

The record may include the value of the sub-segment, which is according to the first set of definitions.

The record may further include the index of the sub-segment indicating sequential position within the sequence of sub-segments of the segment of the message.

The record may include one or more additional sub-segment preceding and/or following the sub-segment according to the sequence of sub-segment defined for the segment. The record may include all sub-segments of the segment, optionally according to their sequential order within the segment.

The record may include the ground truth label, which is according to the second set of definitions.

At 314, features described with reference to 302-312 are iterated for creating multiple records. The multiple records are included in one or more training datasets.

The iterations may be performed for creating records from messages of different types, such as including different segments, different values for sub-segments, and/or originating and/or destined for different components connected to the medical network. The messages may be a representative sample of messages of the medical network, for example, randomly sampled, and/or sampled according to a selected distribution likely representing a statistical sample. Multiple messages may be used for training, such as to avoid ambiguities that may exist in a single messages.

Optionally, a respective training dataset is created for a set of records of a specific type of segment. A different training dataset may be created for each type of segment. For example, about 130 different training datasets corresponding to the about 130 different types of segments defined by HL7 may be created.

At 316, one or more classifiers are trained on the training dataset(s).

Optionally, multiple classifiers are trained, where each classifier is trained on a different training dataset corresponding to a different respective type of segment of the message.

Alternatively or additionally, a classifier is trained on a training dataset of multiple types of segments.

The classifier(s) may be based on an architecture suitable for receiving text and/or other corresponding representation as input, and outputting a classifier category and/or other corresponding representation.

Exemplary architectures include neural networks of various architectures (e.g., convolutional, fully connected, deep, encoder-decoder, recurrent, transformer, graph), a pipeline combination of classifier(s), statistical classifiers and/or other statistical models, support vector machines (SVM), logistic regression, k-nearest neighbor, decision trees, boosting, random forest, a regressor, and/or any other commercial or open source package allowing regression, classification, dimensional reduction, supervised, unsupervised, semi-supervised, and/or reinforcement learning. Machine learning models may be trained using supervised approaches and/or unsupervised approaches.

In an exemplary implementation the classifier(s) is based on a transformer architecture, optionally an adaptation of bidirectional encoder representation from transformers (BERT). The classifier based on the BERT architecture may be adapted from the base architecture with 12 transformer layers and an inner-output dimension of 768. After the transformer layers, global pooling may be included to get one vector with 768 dimensions. Then a fully-connected layer with the number of outputs equal to the number of categories in the specific segment may be included.

Referring now back to FIG. 4, segment 402 is an example of a segment complying with HL7. Segment 402 includes multiple sub-segments 404, which may be parsed as described herein. The sub-segments may be fed into the classifier(s) and/or used for training the classifier(s), as described herein.

Various embodiments and aspects of the present invention as delineated hereinabove and as claimed in the claims section below find experimental and/or calculated support in the following examples.

### EXAMPLES

Reference is now made to the following examples, which together with the above descriptions illustrate some embodiments of the invention in a non limiting fashion.

In a first example based on FIG. 2, a first message is obtained from a medical network.

At 204, the message (denoted m1) which is obtained includes the segment OBR:
OBR|1|3400355735165062|3400355735165062|||||||||||||||||||20221228165556||F

At 206, the message is processed by dividing each segment into corresponding sub-segments, and converting to text, which generates the following in the format (position: text):
1: 1
2: 3400355735165062
3: 3400355735165062
22: 20221228165556
24: F

At 208, each sub-segment is fed into the classifier.

At 210, a probability matrix is computed as described herein from the outcomes of the classifier. Since the OBX segment has 49 possible sub-segments, the probability matrix has 49 columns.

Referring now back to FIG. 5, probability matrix 502 is presented. For simplicity and clarity of explanation, only columns where at least one entry is higher than 0.1 are shown.

At 212, probability matrix 502 of FIG. 5 is analyzed.

Line 1—sub segment 1 (504): since the maximal value of the first line corresponds to the first column (514), the process decides the first sub-segment corresponds to the Set ID sub-segment 514.

Line 2 and Line 3—sub-segment 2 (506) and sub-segment 3 (508): Note that both entries are equal one to another. However, since they have different initial locations, they yield different sets of probabilities (it is noted that in the matrix of probabilities 502, probabilities of columns 2 (516) and 3 (518) computed for lines 2 (506) and 3 (508) are different one from another). However, the probabilities are close enough such that classification of these two sub-segments 506 and 508 is ambiguous. It can be either the “Placer Order Number” sub-segment (516) or the “Filler Order Number” sub-segment (518). But it can't be determined which is which.

Note that, for on-line applications, it doesn't necessarily matter. Since both entries are equal, the value 3400355735165062 may be decoded to both “Placer Order Number” sub-segment (516), and “Filler Order Number” sub-segment (518).

Line 4—sub-segment 22 (510), the highest probability appears at the column 22 (520) which is the “Results Rpt/Status Chng—Date/Time” sub-segment.

Line 5—sub-segment 24 (512), the highest probability appears at the column 25 (522) which is the “Result Status” sub-segment.

Another message denoted m2 is obtained from the same medical network as m1. The second message is processed using features described with reference to FIG. 2.

At 204, m2 is obtained from the same medical network as m1:
OBR|5|E1239494|3404923749823749|||||||||||||||||||20231245324321||A

At 206, m2 is processed by dividing each segment into corresponding sub-segments, and converting to text.

At 208, each sub-segment is fed into the classifier.

At 210, applying a process similar to the process described with reference to m1, another probability matrix is obtained for m2 from the outcomes of the classifier.

Referring now back to FIG. 6, probability matrix 602 is presented. For simplicity and clarity of explanation, only columns where at least one entry is higher than 0.1 are shown.

At 212, probability matrix 602 of FIG. 6 is analyzed.

Line 1—sub segment 1 (604): since maximal value of the first line corresponds to the first column 614, the process decides the first sub-segment corresponds to the Set ID sub-segment 614.

Line 2 and 3 sub-segments 2 (606) and 3 (608)—Now the ambiguity between these two sub-segments 606 and 608 may be resolved. Line 2 (606) is mapped to column 2 (616) and Line 3 (608) is mapped to column 3 (618).

Line 4—sub-segment 22 (610), the highest probability appears at the column 22 (620) which is “Results Rpt/Status Chng—Date/Time” sub-segment.

Line 5—sub-segment 24 (612), the highest probability appears at the column 25 (622) which is of “Result Status” sub-segment.

At 214, the mapping dataset is generated from the resolved probability table 502 of FIG. 5 and from the resolved probability table 602 of FIG. 6, where a single column is mapped to a single row, i.e., a single classification category is selected for each sub-segment.

Referring now back to FIG. 7, mapping dataset 702 defines a mapping for sub-segments between the first set of definitions 704 and the second set of definitions 706, as described herein.

In a second example based on FIG. 2, a third message denoted m3 is obtained from another medical network different from the medical network of the first example:

At 204, the message (m3) which is obtained includes the following segment:
obr|||||ME10165\hat{ }XR ELBOW\hat{ }LE\hat{ }|||||||Left elbow test\hat{ }.\hat{ }|XR ELBOW\hat{ }LE\hat{ }|||E6200|||FKRI3492|||||Y1119|||\hat{ }\hat{ }\hat{ }20210812163000\hat{ }

At 206, the message is processed by dividing each segment into corresponding sub-segments, and converting to text, which generates the following in the format (position: text):
5: ME10165\hat{ }XR ELBOW\hat{ }LE\hat{ }
12: Left elbow test\hat{ }.\hat{ }
13: XR ELBOW\hat{ }LE\hat{ }
16: E6200
19: FKRI3492
24: Y1119
27: \hat{ }\hat{ }\hat{ }20210812163000\hat{ }

At 208, each sub-segment is fed into the classifier.

At 210, a probability matrix is computed as described herein from the outcomes of the classifier. Since the OBX segment has 49 possible sub-segments, the probability matrix has 49 columns.

Referring now back to FIG. 8, probability matrix 802 is presented. For simplicity and clarity of explanation, only columns where at least one entry is higher than 0.1 are shown.

At 212, probability matrix 802 of FIG. 8 is analyzed.

Line 1 sub-segment 5 (804): the highest probability appears at the column 4 (818).

Line 2 sub-segment 12 (806): the highest probability appears at the column 30 (826).

Line 3 sub-segment 13 (808): the highest probability appears at the column 13 (820).

Line 4 sub-segment 19 (810): the highest probability appears at the column 3 (816).

Line 5 sub-segment 24 (812): the highest probability appears at the column 24 (822).

Line 6 sub-segment 27 (814): the highest probability appears at the column 27 (824).

At 214, the mapping dataset is generated from the resolved probability table 802 of FIG. 8, where a single column is mapped to a single row, i.e., a single classification category is selected for each sub-segment.

It is clear that, for this medical network, the sub-segments definition significantly deviates from the standard recommendations.

Referring now back to FIG. 9, mapping dataset 902 defines a mapping for sub-segments between the first set of definitions 904 and the second set of definitions 906, as described herein.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant standards for communication of health related information will be developed and the scope of the term standard is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A computer implemented method of supporting automatic integration of an interface of a medical component within a medical network, comprising:
monitoring at least one message within the medical network complying with a standard for communication of health related data according to a first set of definitions of segments and sub-segments of the messages;
feeding at least one sub-segment of a segment of the at least one message into at least one classifier;
obtaining at least one classification category for each of the at least one sub-segments as an outcome of the at least one classifier,
wherein the at least one classification category is according to a second set of definition of sub-segments of messages destined for the interface of the medical component being integrated within the medical network;
generating a mapping dataset according to the at least one classification category obtained for each of the at least one sub-segments of the at least one message, for mapping between the first set of definitions and the second set of definitions; and
automatically converting sub-segments of messages sent between the medical network and the interface according to the mapping dataset.

2. The computer implemented method of claim 1, wherein the standard for communication of health related data comprises Health Level 7 (HL7), and the segments and sub-segments are defined by HL7.

3. The computer implemented method of claim 1, wherein the at least one classifier is trained on a training dataset of a plurality of records, wherein a record includes a sub-segment of a segment of a message according to the first set of definitions and a ground truth label according to the second set of definitions.

4. The computer implemented method of claim 3, wherein the record further includes an index of the sub-segment indicating sequential position within a plurality of sub-segments of the segment of the message.

5. The computer implemented method of claim 3, wherein the record further includes at least one additional sub-segment preceding and/or following the sub-segment according to a sequence of sub-segment defined for the segment.

6. The computer implemented method of claim 1, wherein a plurality of classifiers are trained, each classifier trained on a different training dataset for a different respective segment of a plurality of segments of the message, each training dataset including a plurality of records, wherein a record includes a sub-segment of the respective segment of a message according to the first set of definitions and a ground truth label according to the second set of definitions.

7. The computer implemented method of claim 1, further comprising:
parsing each message into a plurality of segments;
for each respective segment of the plurality of segments of the message:
identifying a type of a plurality of types for the respective segment,
selecting a classifier of a plurality of classifiers according to the type of the respective segment,
wherein each classifier is trained for generating the at least one classification category in response to an input of at least one sub-segment of the segment satisfying a specific type of the plurality of types, wherein a plurality of classifiers are trained for the plurality of segment types,
wherein the respective sub-segment of the segment is fed into the selected classifier.

8. The computer implemented method of claim 1, wherein feeding comprises feeding a combination of a sub-segment of a segment of the message and an index of the sub-segment indicating sequential position within a plurality of sub-segments of the segment of the message.

9. The computer implemented method of claim 1, wherein feeding comprises feeding a combination of a sub-segment of the message and at least one additional sub-segment preceding and/or following the sub-segment according to a sequence of sub-segments of a segment of the message.

10. The computer implemented method of claim 1, wherein the outcome of the at least one classifier fed a sub-segment of the first set of definitions comprises a plurality of probabilities for a plurality of sub-segments based on the second set of definitions, wherein the at least one classification category for the sub-segment fed into the at least one classifier is computed by applying a process to the plurality of probabilities of the plurality of sub-segments, wherein the process is fed a matrix of the plurality of probabilities and outputs a unique classification category for all non-zero sub-segments.

11. The computer implemented method of claim 10, wherein the process selects a definition from the second set having a highest probability, wherein the at least one classification category comprises the selected definition.

12. The computer implemented method of claim 10, wherein the process selects a definition from the second set having a highest probability when a difference between the highest probability and second highest probability is greater than a threshold.

13. The computer implemented method of claim 1, further comprising: in response to an indicating that no valid classification of the sub-segment is determined, generating a presentation on a display for manual classification of the sub-segment by a user.

14. The computer implemented method of claim 1, wherein the monitoring, the feeding, the obtaining, and the generating are performed during a set-up phase where the monitoring is performed on messages sent between existing network connected components over the medical network excluding the interface, and the automatic conversion is performed dynamically in real-time for messages exchanged between the medical network and the interface.

15. The computer implemented method of claim 14, further comprising iterating the set-up phase periodically at a plurality of time intervals and/or in response to an event, for regenerating the mapping dataset and/or detecting changes in the mapping dataset.

16. The computer implemented method of claim 1, wherein the monitoring, the feeding, the obtaining, and the generating are performed in real-time for each message exchanged between the medical network and the interface for dynamic computation of a mapping dataset for each message, wherein each message is dynamically converted in real-time using the mapping dataset computed for each respective message.

17. The computer implemented method of claim 1, wherein the interface comprises an input into a machine learning model.

18. The computer implemented method of claim 1, further comprising, for each message:
dividing each segment of a plurality of segments of the message into a plurality of sub-segments;
converting each sub-segment into a string;
wherein feeding comprises feeding the string.

19. The computer implemented method of claim 1, wherein components connected to the medical network are selected from: electronic medical record (EMR) dataset, imaging device, and AI application.

20. The computer implemented method of claim 1, wherein the medical network comprises an integration target and the medical component comprises an integration source.

21. The computer implemented method of claim 1, wherein the second set of definitions are of a translator process, and the interface of the medical component complies with a third set of definitions;
wherein the mapping dataset comprises a first mapping dataset for mapping between the first set of definitions of the medical network and the second set of definitions of the translator, and
further comprising performing the monitoring for at least one message according to the third set of definitions, the feeding into at least one second classifier, the obtaining from the at least one second classifier, and the generating a second mapping dataset for mapping between the second set of definitions of the translator process and the third set of definitions of the interface,
wherein automatically converting comprises automatically converting messages sent between the medical network and the interface according to the first mapping dataset and the second mapping dataset, by converting from the first definition to the second definition, and from the second definition to the third definition, and/or from the third definition to the second definition, and from the second definition to the first definition.

22. A system for supporting automatic integration of an interface of a medical component within a medical network, comprising:
at least one processor executing a code for:
monitoring at least one message within the medical network complying with a standard for communication of health related data according to a first set of definitions of segments and sub-segments of the messages;
feeding at least one sub-segment of a segment of the at least one message into at least one classifier;
obtaining at least one classification category for each of the at least one sub-segments as an outcome of the at least one classifier,
wherein the at least one classification category is according to a second set of definition of sub-segments of messages destined for the interface of the medical component being integrated within the medical network;
generating a mapping dataset according to the at least one classification category obtained for each of the at least one sub-segments of the at least one message, for mapping between the first set of definitions and the second set of definitions; and
automatically converting sub-segments of messages sent between the medical network and the interface according to the mapping dataset.

23. A non-transitory medium storing program instructions for supporting automatic integration of an interface of a medical component within a medical network, which when executed by at least one processor, cause the at least one processor to:
monitor at least one message within the medical network complying with a standard for communication of health related data according to a first set of definitions of segments and sub-segments of the messages;
feed at least one sub-segment of a segment of the at least one message into at least one classifier;
obtain at least one classification category for each of the at least one sub-segments as an outcome of the at least one classifier,
wherein the at least one classification category is according to a second set of definition of sub-segments of messages destined for the interface of the medical component being integrated within the medical network;
generate a mapping dataset according to the at least one classification category obtained for each of the at least one sub-segments of the at least one message, for mapping between the first set of definitions and the second set of definitions; and
automatically convert sub-segments of messages sent between the medical network and the interface according to the mapping dataset.
